(19) United States
(12) Patent Application Publication
(10) Pub. No.: US 2015/0024472 A1
(43) Pub. Date: Jan. 22, 2015

(54) BIOLOGICAL GROWTH MONITORING AND PARAMETER EXTRACTION SYSTEM

(71) Applicant: National Taiwan University, Taipei (TW)

(72) Inventors: Jui-Jen Chou, Taipei (TW); Mei-Wen Fang, Taipei (TW); Ming-Yen Lin, Taipei (TW)

(73) Assignee: National Taiwan University, Taipei (TW)

(21) Appl. No.: 14/255,287

(22) Filed: Apr. 17, 2014

(30) Foreign Application Priority Data
Jul. 22, 2013 (TW) 102126069

(51) Int. Cl. C12M 1/34; C12M 1/00; C12M 3/00; C12M 1/36

(52) U.S. Cl. CPC C12M 41/46; C12M 41/48; C12M 23/52; C12M 23/44
USPC 435/286.5; 435/286.1; 435/286.6

(57) ABSTRACT

A biological growth monitoring and parameter extraction system includes a comparison group platform and an experimental group platform. First environmental sensing modules of the comparison group platform extracts first environmental parameters of a biological comparison area, a first control module transmits the parameters to the experimental group platform, and a first biological inspection module extracts growth conditions of organisms of the biological comparison area. A biological experiment area of the experimental group platform emulates growth environment of the biological comparison area, second environmental sensing modules extract second environmental parameters of the biological experiment area, and the first environmental parameters are used as reference inputs of the experimental group platform, so as to enable the second control module to obtain an error value by comparing the second and the first environmental parameters.

### FIELD OF THE INVENTION

The present invention relates to a biological growth monitoring and parameter extraction system, and more particularly, to a biological growth monitoring and parameter extraction system with a comparison group platform and an experimental group platform.

### BACKGROUND OF THE INVENTION

In the literature or publicly available information concerning biological growth, there have been disagreements on how to obtain better biological growth parameters, resulting in lots of user confusions. The reason behind this is lack of observability and controllability during the process of finding better biological growth parameters.

Furthermore, the biological growth state cannot be identified in real-time using the traditional approach. Therefore, it is not easy to find the relationships between the environmental parameters and the growth state of the organisms. A longer testing period is usually required to find the relationship. As a result, real-time control with the better environmental parameters is not possible.

In addition, it is more difficult to obtain better biological growth parameters for a mass-production system, mainly due to the facts that the scale of the system is large, and input costs are high. Designing multiple test conditions and running individual test on each of the units are not feasible, because there may be considerable losses if it fails.

Also, traditionally, environmental parameters of the wind, light, water, nutrient, and air of the overall environment are monitored by environmental sensors during the experiments. The sensors are not disposed in proximity to the organisms. Spatial environmental parameters are not uniform very often, so the measured environmental parameters may not be the actual environmental parameters around the organisms, resulting in inconsistencies in the relationships between the biological growth parameters obtained and the growth states of the organisms.

Therefore, there is need to provide a biological growth monitoring and parameter extraction system to overcome the above prior-art problems.

### SUMMARY OF THE INVENTION

In light of the foregoing drawbacks, the present invention provides a biological growth monitoring and parameter extraction system, which includes a comparison group platform and an experimental group platform. The comparison group platform may include a biological comparison area, a plurality of first environmental sensing modules, a first biological inspection module and a first control module. The first environmental sensing modules are used for extracting a plurality of first environmental parameters of the biological comparison area. The first biological inspection module is used for extracting a growth state of a first organism of the biological comparison area, and the first control module is used for transmitting the first environmental parameters. The experimental group platform may include a biological experiment area, a plurality of second environmental sensing modules, a second biological inspection module, a second control module and a plurality of actuator modules. The biological experiment area is used for emulating the growth environment of the biological comparison area. The second environmental sensing modules are used for extracting a plurality of second environmental parameters of the biological experiment area. The second biological inspection module is used for extracting a growth state of a second organism of the biological experiment area, and the first environmental parameters are used as reference inputs for the experimental group platform, so that the second control module obtains an error value by comparing the first environmental parameters with the second environmental parameters. When the error value exceeds a predetermined allowable range, the second control module controls the actuator modules to adjust the corresponding second environmental parameters to lower the error value to be within the allowable range.

When the experimental group platform experiments on the second environmental parameters, the second control module controls the actuator modules to adjust one of the second environmental parameters, while leaving the rest of the second environmental parameters unchanged, so as to know how the adjusted second environmental parameter affects the growth state of the second organism of the biological experiment area, allowing the second environmental sensing modules to obtain a preferred value of the adjusted second environmental parameters

The experimental group platform may further include a growth chamber in which the biological experiment area, the second environmental sensing modules, the second biological inspection module and the actuator modules are disposed.

The first biological inspection module extracts the growth state of the first organism in real-time to inspect the growth quality of the first organism. The second biological inspection module extracts the growth state of the second organism in real-time to inspect the growth quality of the second organism.

Based on the relationship between the first environmental parameters extracted by the first environmental sensing modules and the growth quality of the first organism inspected by the first biological inspection module, and the relationship between the second environmental parameters extracted by the second environmental sensing modules and the growth quality of the second organisms inspected by the second biological inspection module, the second control module combined with the expert knowledge bank identifies more suitable second environmental parameters.

The first environmental sensing modules or the second environmental sensing modules may be light sensors, temperature sensors, humidity sensors, carbon dioxide sensors or nutrient concentration sensors. The actuator modules may be light controlled components, temperature controlled components, humidity controlled components, carbon dioxide controlled components, nutrient controlled components, or nutrient circulation controlled components.

The biological growth monitoring and parameter extraction system may further include a remote control platform connected to the comparison group platform and the experimental group platform via a network for remotely controlling the comparison group platform and the experimental group platform. The remote control platform may include a communication module and a control module. The communication module performs remote communication with the comparison group platform and the experimental group platform, so that the control module remotely manipulates the first control module, the second control module and the actuator modules, and extracts the first environmental parameters, the growth state of the first organism, the second environmental parameters, and the growth state of the second organism.

The biological growth monitoring and parameter extraction system may further include an expert knowledge bank connected to the remote control platform for analyzing and storing the first environmental parameters of the biological comparison area, the growth state of the first organism, the second environmental parameters of the biological experiment area, the growth state of the second organism, the preferred second environmental parameters.

The biological growth monitoring and parameter extraction system may further include a mobile trolley for disposing the biological experiment area, the second environmental sensing modules, the second biological inspection module, the second control module and the actuator modules of the experimental group platform thereon.

From the above, it is clear that the biological growth monitoring and parameter extraction system mainly includes the comparison group platform and the experimental group platform. The growth environment of the biological comparison area of the comparison group platform can be emulated by the biological experiment area of the experimental group platform. This is achieved through forming an environmental parameter feedforward control system by feeding forward the environmental parameters of the comparison group platform to the experimental group platform as reference inputs. By placing the environmental sensing modules of the experimental group platform in proximity to the biological experiment area, feedback control of the environmental parameters of the experimental group platform is carried out, and the growth states of the organisms are extracted in real-time by the biological inspection modules.

Accordingly, the present invention can adjust the environmental parameters of the experimental group platform in real-time; inspect the growth states of the organisms of the comparison group platform and the experimental group platform in real-time; and identify the effects and relationships of the environmental parameter on the growth of the organisms in real-time, so as to obtain environmental parameters more suitable for growth of the organism and establish an expert knowledge bank.

Moreover, the present invention monitors the various modules of the comparison group platform and the experimental group platform in real-time, analyzes and stores information such as the first environmental parameters of the biological comparison area, the growth state of the first organisms, the second environmental parameters of the biological experiment area, the growth state of the second organism(s), and the preferred or more suitable second environmental parameters.

### BRIEF DESCRIPTION OF THE DRAWINGS

The present invention can be more fully understood by reading the following detailed description of the preferred embodiments, with reference made to the accompanying drawings, wherein:

FIG. 1 is a diagram illustrating the feedforward and feedback of a biological growth monitoring and parameter extraction system in accordance with the present invention;

FIG. 2 is a block diagram illustrating a biological growth monitoring and parameter extraction system with an expert knowledge bank and a remote control platform in accordance with the present invention; and

FIG. 3 is a perspective view showing a mobile trolley, a growth chamber and the remote control platform of the biological growth monitoring and parameter extraction system in accordance with the present invention.

### DETAILED DESCRIPTION OF THE EMBODIMENTS

Implementations of the present invention are described by the following specific embodiments. Those with ordinary skills in the arts can readily understand the other advantages and functions of the present invention from the disclosure of this specification.

It should be noted that the structures, proportions, sizes and the like shown in the attached drawings are to be considered only in conjunction with the contents of this specification to facilitate understanding and reading of those skilled in the art, and are not intended to limit the scope of present invention, so they do not hold any real technically significance, and any changes or modifications in the structures, the proportions, the sizes and the like should fall within the scope of the technical contents disclosed in the present invention as long as they do not affect the effects and the objectives achieved by the present invention.

Meanwhile, terms such as “above”, “one”, “first”, “second” and so on used herein are for illustration purposes only, and not intended to limit the scope of the present invention in any way, any changes or modifications of the relative relationships of elements are therefore to be construed as within the scope of the present invention as long as no substantial changes are made to the technical contents.

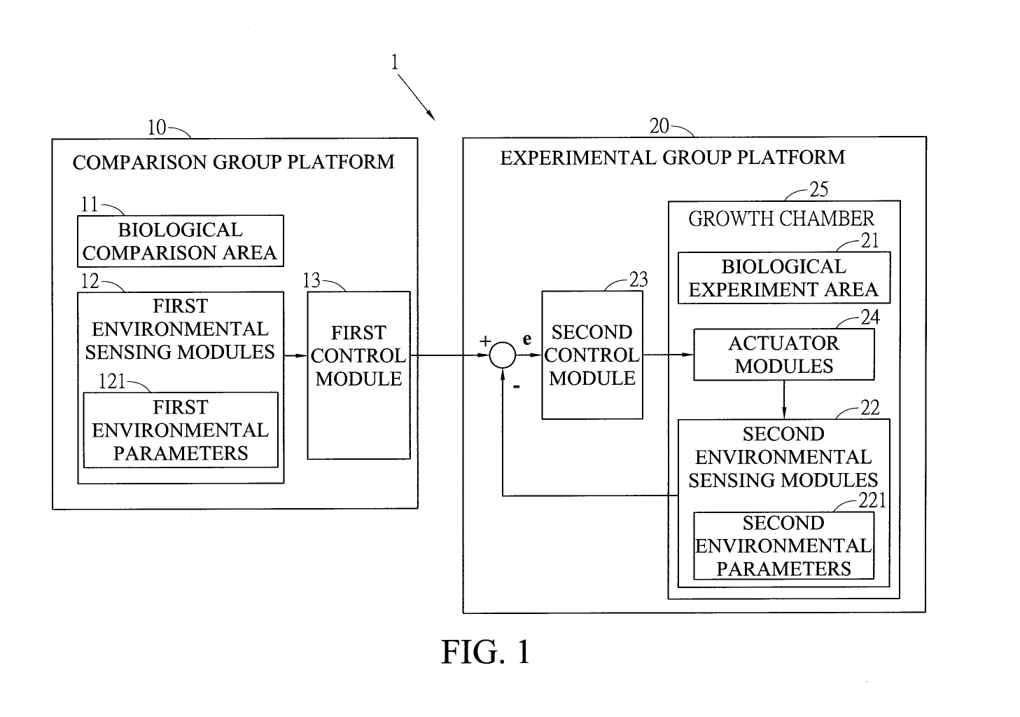

FIG. 1 is a diagram illustrating the feedforward and feedback of a biological growth monitoring and parameter extraction system in accordance with the present invention. As shown, the biological growth monitoring and parameter extraction system 1 includes a comparison group platform 10 and an experimental group platform 20.

The comparison group platform 10 includes a biological comparison area 11, a plurality of first environmental sensing modules 12, a first control module 13 and a first biological inspection module (not shown).

The biological comparison area 11 can be an organism mass-production area for nurturing a large number of first organisms such as plants, animals or micro-organisms.

The first environmental sensing modules 12 are used for extracting a plurality of first environmental parameters 121 of the biological comparison area 11. The first environmental parameters 121 are received and transmitted by the first control module 13 to the experimental group platform 20.

The experimental group platform 20 includes a biological experiment area 21, a plurality of second environmental sensing modules 22, a second control module 23, a plurality of actuator modules 24 and a second biological inspection module (not shown).

The biological experiment area 21 can be an area with a single organism or a few organisms for nurturing one or a few second organisms the same as the first organisms. Meanwhile, the biological experiment area 21 emulates the growth environment of the biological comparison area 11, for example, with the same or very similar growth conditions such as light, temperature, humidity, carbon dioxide or nutrient concentrations. Light may include light quality (optical frequency), the amount of light (illumination) or photoperiod.

The second environmental sensing modules 22 are provided in the proximity of the second organism(s) of the biological experiment area 21 for extracting second environmental parameters 221 of the biological experiment area 21. The first environmental parameters 121 are used as reference inputs for the experimental group platform 20, so as to enable the second control module 23 to obtain an error value e by comparing the second environmental parameters 221 and the first environmental parameters 121. When the error value e exceeds a predetermined allowable range, the second control module 23 controls the actuator modules 24 to adjust the second environmental parameters accordingly, so as to control the error value e to be within the allowable range.

In other words, the first environmental parameters 121 can be regarded as the target environmental parameters, and are compared with the second environmental parameters 221 to obtain the error value e. Thereafter, the second control module 23 then decides whether to activate the actuator modules 24 in order to control the second environmental parameters 221 based on the error value e, thereby forming an environmental parameter feedforward and feedback control system.

When the experimental group platform 20 performs experiments on the second environmental parameters 221, the second control module 23 will control the actuator modules 24 to adjust one of the second environmental parameters 221 while leaving the rest of the second environmental parameters 221 unchanged, so as to know the level which the adjusted second environmental parameters 221 affects the growth of the second organism(s) of the biological experiment area 21, thereby allowing the second environmental sensing modules 22 to obtain a preferred value of the adjusted second environmental parameters 221.

The experimental group platform 20 may include a growth chamber 25. The biological experiment area 21, the second environmental sensing modules 22, the actuator modules 24 and the second biological inspection module can be provided in the growth chamber 25 so as to monitor the wind, the light, the water, the nutrient, and the air of the second organism of the biological experiment area 21.

The above first environmental sensing modules 12 or the second environmental sensing modules 22 can be light sensors, temperature sensors, humidity sensors, carbon dioxide sensors, nutrient concentration sensors or the like. The first environmental parameters 121 can be illumination, optical frequency, photoperiod, temperature, humidity, carbon dioxide, nutrient concentration or the like.

The first control module 13 can be a controller, a control program, a receiving module or a transmission module. The second control module 23 can be a controller, a control program, a comparator, a receiving module or a transmission module.

Each of the actuator modules 24 corresponds to a second environmental sensing module 22, and can be a light controlled component, a temperature controlled component, a humidity controlled component, a carbon dioxide controlled component, a nutrient controlled component, or a nutrient circulation controlled component.

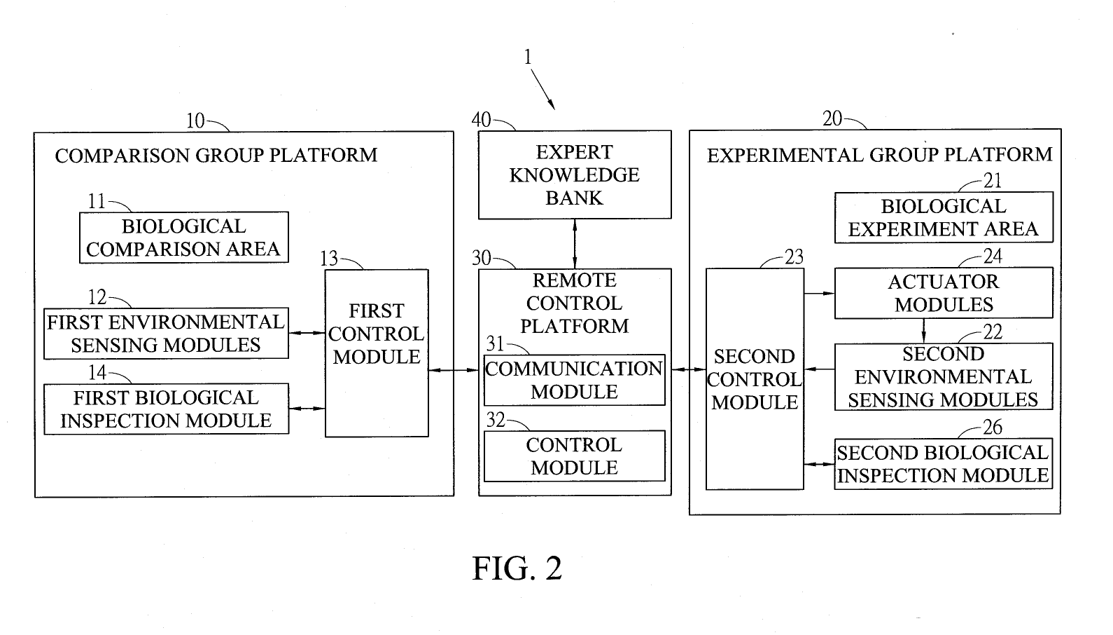

FIG. 2 is a block diagram illustrating a biological growth monitoring and parameter extraction system with an expert knowledge bank and a remote control platform in accordance with the present invention. As shown, the biological growth monitoring and parameter extraction system 1 also includes a remote control platform 30 and an expert knowledge bank 40, in addition to the comparison group platform 10 and the experimental group platform 20 shown in FIG. 1.

The comparison group platform 10 and the experimental group platform 20 shown in FIG. 2 are similar to those shown in FIG. 1, and the main differences are illustrated as follow.

In FIG. 2, the comparison group platform 10 includes a first biological inspection module 14, such as an image retrieving module for real-time retrieving the growth state of the first organisms of the biological comparison area 11, such that the biological growth monitoring and parameter extraction system 1 automatically inspects and records the growth quality of the first organisms. The experimental group platform 20 also includes a second biological inspection module 26, such as an image retrieving module, for real-time retrieving the growth state of the second organism(s) of the biological experiment area 21, such that the biological growth monitoring and parameter extraction system 1 automatically inspects and records the growth quality of the second organism(s). As such, the differences, associations, and effects of the growth states between the first and the second organisms can be identified based on the first environmental parameters 121 and the second environmental parameters 221, so as to adjust the second environmental parameters 221 in real-time.

Therefore, based on the relationship between the first environmental parameters 121 extracted by the first environmental sensing modules 12 and the growth quality of the first organisms inspected by the first biological inspection module 14, and the relationship between the second environmental parameters 221 extracted by the second environmental sensing modules 22 and the growth quality of the second organisms inspected by the second biological inspection module 26, the second control module 23 and the expert knowledge bank 40 may identify preferred or more suitable second environmental parameters 221.

The remote control platform 30 can be connected to the comparison group platform 10 and the experimental group platform 20 via a wired network or a wireless network for remotely controlling the comparison group platform 10 and the experimental group platform 20.

More specifically, the remote control platform 30 may include a communication module 31 and a control module 32. The communication module 31 performs remote communication with the first control module 13 of the comparison group platform 10 and the second control module 23 of the experimental group platform 20, so that the control module 32 can remotely manipulate the first control module 13, the second control module 23 and the actuator modules 24, and extract information such as the first environmental parameters 121, the growth state of the first organisms, the second environmental parameters 221, and the growth state of the second organism(s).

The expert knowledge bank 40 is connected to the remote control platform 30 so as for users to analyze, predict and store information such as the growth state of the first organisms of the biological comparison area 11, the first environmental parameters 121, the growth state of the second organism(s) of the biological experiment area 21, the second environmental parameters 221 and the preferred or more suitable second environmental parameters 221, which can be used as a reference for nurturing the first organisms by the users or others in the future.

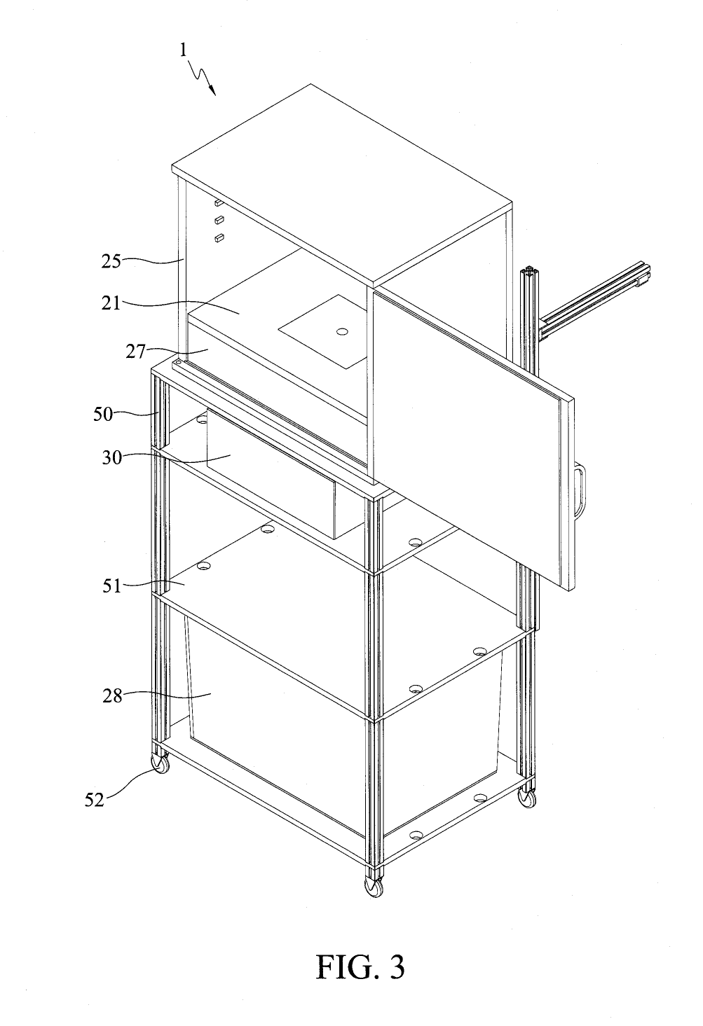

FIG. 3 is a perspective view showing a mobile trolley, the growth chamber and the remote control platform of the biological growth monitoring and parameter extraction system in accordance with the present invention.

As shown, the biological growth monitoring and parameter extraction system 1 may include a mobile trolley 50. The mobile trolley 50 includes a plurality of shelves 51 and a plurality of pulleys 52. The biological experiment area 21 of the experimental group platform 20, the second environmental sensing modules 22, the second control module 23, the actuator modules 24, the growth chamber 25, the second biological inspection module 26 and the remote control platform 30 shown in FIGS. 1 and 2 can be provided on the shelves 51, and an interconnected nutrient supply tray 27 and a circulating nutrient supply tray 28 can also be provided. The pulleys 52 are used for moving the shelves 51 of the mobile trolley 50 and the various objects thereon.

In summary, the biological growth monitoring and parameter extraction system mainly includes the comparison group platform and the experimental group platform. The growth environment of the biological comparison area of the comparison group platform can be emulated by the biological experiment area of the experimental group platform. This is achieved through forming an environmental parameter feedforward control system by feeding forward the environmental parameters of the comparison group platform to the experimental group platform as reference inputs. By placing the environmental sensing modules of the experimental group platform in the proximity of the biological experiment area, the feedback control of the environmental parameters of the experimental group platform is carried out, and the growth states of the organisms are extracted in real-time by the biological inspection modules.

Accordingly, the present invention can adjust the environmental parameters of the experimental group platform in real-time, inspect the growth states of the organisms of the comparison group platform and the experimental group platform in real-time, and identify the effects and relationships of the environmental parameter on the growth of the organisms in real-time, so as to obtain environmental parameters more suitable for growth of the organism and establish an expert knowledge bank.

Meanwhile, the present invention monitors the various modules of the comparison group platform and the experimental group platform in real-time, and analyzes and stores information such as the first environmental parameters of the biological comparison area, the growth state of the first organisms, the second environmental parameters of the biological experiment area, the growth state of the second organism(s), and the preferred or more suitable second environmental parameters.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A biological growth monitoring and parameter extraction system, comprising:
a comparison group platform comprising a biological comparison area, a plurality of first environmental sensing modules for extracting a plurality of first environmental parameters of the biological comparison area, a first biological inspection module for extracting a first growth state of a first organism of the biological comparison area, and a first control module for transmitting the first environmental parameters; and
an experimental group platform comprising a biological experiment area for emulating the growth environment of the biological comparison area, a plurality of second environmental sensing modules for extracting a plurality of second environmental parameters of the biological experiment area, a second biological inspection module for extracting a second growth state of a second organism of the biological experiment area, a second control module and a plurality of actuator modules, wherein the first environmental parameters are used as reference inputs for the experimental group platform, so that the second control module obtains an error value by comparing the first environmental parameters and the second environmental parameters, and when the error value exceeds a predetermined allowable range, the second control module controls the actuator modules to adjust corresponding second environmental parameters so as to control the error value to be within the predetermined allowable range.

2. The biological growth monitoring and parameter extraction system of claim 1, wherein when the experimental group platform performs experiments on the second environmental parameters, the second control module controls the actuator modules to adjust one of the second environmental parameters, while leaving the rest of the second environmental parameters unchanged, so as to obtain a level which the one of the second environmental parameters affects the second growth state of the second organism of the biological experiment area, so as for the second environmental sensing modules to obtain a preferred value of the one of the second environmental parameters

3. The biological growth monitoring and parameter extraction system of claim 1, wherein the experimental group platform further comprises a growth chamber in which the biological experiment area, the second environmental sensing modules, the second biological inspection module and the actuator modules are disposed.

4. The biological growth monitoring and parameter extraction system of claim 1, wherein the first biological inspection module extracts the first growth state of the first organism in real-time to inspect growth quality of the first organism, and the second biological inspection module extracts the second growth state of the second organism in real-time to inspect growth quality of the second organism.

5. The biological growth monitoring and parameter extraction system of claim 4, wherein based on a first relationship between the first environmental parameters extracted by the first environmental sensing modules and the growth quality of the first organism inspected by the first biological inspection module, and a second relationship between the second environmental parameters extracted by the second environmental sensing modules and the growth quality of the second organisms inspected by the second biological inspection module, the second control module or an expert knowledge bank identifies more suitable second environmental parameters.

6. The biological growth monitoring and parameter extraction system of claim 1, wherein the first environmental sensing modules or the second environmental sensing modules are light sensors, temperature sensors, humidity sensors, carbon dioxide sensors or nutrient concentration sensors, the actuator modules are light controlled components, temperature controlled components, humidity controlled components, carbon dioxide controlled components, nutrient controlled components, or nutrient circulation controlled components.

7. The biological growth monitoring and parameter extraction system of claim 1, further comprising a remote control platform connected to the comparison group platform and the experimental group platform via a network for remotely controlling the comparison group platform and the experimental group platform.

8. The biological growth monitoring and parameter extraction system of claim 7, wherein the remote control platform includes a communication module and a control module, and the communication module performs remote communication with the comparison group platform and the experimental group platform, so that the control module remotely manipulates the first control module, the second control module and the actuator modules, and extracts the first environmental parameters, the first growth state of the first organism, the second environmental parameters, and the second growth state of the second organism.

9. The biological growth monitoring and parameter extraction system of claim 7, further comprising an expert knowledge bank connected to the remote control platform for analyzing and storing the first environmental parameters of the biological comparison area, the first growth state of the first organism, the second environmental parameters of the biological experiment area, the second growth state of the second organism, and a preferred one of the second environmental parameters.

10. The biological growth monitoring and parameter extraction system of claim 1, further comprising a mobile trolley for disposing the biological experiment area, the second environmental sensing modules, the second biological inspection module, the second control module and the actuator modules of the experimental group platform thereon.
